(19) United States
(12) Patent Application Publication
(10) Pub. No.: US 2022/0175721 A1
(43) Pub. Date: Jun. 9, 2022

(54) TREATMENT FOR POLYCYSTIC OVARIAN SYNDROME (PCOS)

(71) Applicant: Cadila Healthcare Limited, Ahmedabad, Gujarat (IN)

(72) Inventors: Mukul Jain, Ahmedabad, Gujarat (IN); Deven V. Parmar, Ahmedabad, Gujarat (IN); Amit Joharapurkar, Ahmedabad, Gujarat (IN)

(21) Appl. No.: 17/430,381

(22) PCT Filed: Feb. 12, 2020
(86) PCT No.: PCT/IB2020/051118

§ 371 Date: Aug. 12, 2021

(30) Foreign Application Priority Data
Feb. 13, 2019 (IN) 201921005701

(51) Int. Cl. A61K 31/40

(52) U.S. Cl. CPC A61K 31/40

(57) ABSTRACT

Present invention provides the method of treating polycystic ovarian syndrome using compound of formula (I) or pharmaceutically acceptable salt thereof, preferably it provides method of treating polycystic ovarian syndrome in the patient diagnosed with NAFLD. Invention also provides a pharmaceutical composition comprising compound of formula (I) for treatment of polycystic ovarian syndrome, wherein compound of formula (I) is

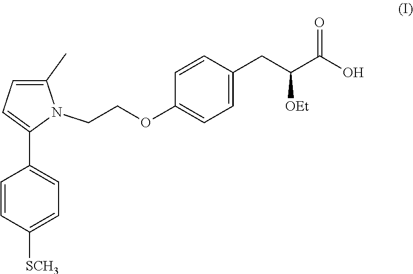 [SMILES: CCO[C@@H](Cc1ccc(OCCn2c(C)ccc2-c2ccc(SC)cc2)cc1)C(=O)O]

### CROSS-REFERENCE TO RELATED APPLICATIONS

This application is a U.S. national stage patent application under 35 U.S.C. § 371 of International Application No. PCT/IB2020/051118, filed on Feb. 12, 2020, which application claims the benefit of and priority to Indian Patent Application No. 201921005701, filed on Feb. 13, 2019, each of which is incorporated by reference herein in its entirety.

### FIELD OF THE INVENTION

The present invention is related to the development of pharmaceutical composition of compound of formula (I) or its pharmaceutically acceptable salts for use in treatment of polycystic ovarian syndrome (PCOS). The present invention provides the use of Compound of formula (I) or its pharmaceutically acceptable salts for the treatment of polycystic ovarian syndrome (PCOS) in patients diagnosed with NAFLD (Non-alcoholic fatty liver disease).

### BACKGROUND OF THE INVENTION

Polycystic ovarian syndrome (PCOS) is a disease occurring in females, and is characterized by hyperandrogenism. PCOS represents a spectrum of phenotypes associated with follicle growth arrest, chronic anovulation, minimal granulosa cell proliferation, hyperthecosis, hyperandrogenemia, central adiposity and insulin resistance. In PCOS, plasma luteinizing hormone (LH) is high and FSH is low, increasing the LH/FSH ratio, typically due to increased LH or GnRH (Gonadotropin releasing hormone) pulse (Semin Reprod Med. 2002 November; 20(4):317-26). In PCOS, disturbances occur in ovarian steroid synthesis, hyperinsulinemia being one of the condition which increases ovarian androgen production and stimulate LH and increases LH/GnRH secretion. A neuroendocrine defect of GnRH secretion leads to persistent hypersecretion of LH in non-obese PCOS patients (Endocrinol Metab Clin North Am. 1999 June; 28(2):295-324). Many females with PCOS suffer from menstrual cycle abnormalities, hirsutism and infertility (Hum Reprod Update. 2015 September; 21(5):575-92).

There are no approved treatments for PCOS (Syst Rev. 2015 Sep. 23; 4(1):125). Optimal first line treatment of PCOS in adolescents remains controversial. Lifestyle changes (dietary and exercise modification) followed by either oral contraceptive pills (OCP) to control symptoms of hyperandrogenism is normally recommended, which has not more than 50% clinical success. However, there is significant variability in clinical practice, depending on whether the physician and patient's primary goal of treatment is to treat the symptoms of hyperandrogenism or the features of metabolic syndrome. Additionally, in clinical practice anti-androgenic medications such as spironolactone, flutamide, and insulin sensitizing agents such as metformin or pioglitazone are used as add-on therapy when OCP or metformin fail to produce the clinically desired outcomes, yet their use in adolescent population is not recommended. Anti-androgens like spironolactone are second choice having risk of electrolyte imbalance. GnRH analogue is the next line of therapy to reduce androgen with lack of potency needing additional hormonal therapy to counteract the chemical menopause that they induce. Metformin may be administered to reduce androgen level, improve menstrual regularity and fertility as well as insulin resistance. Clomiphene with or without metformin improves conception rates with risk of ovarian hyperstimulation and multiple pregnancies (J Clin Endocrinol Metab. 2013 December; 98(12):4565-92).

Increased prevalence of NAFLD has been reported in patients with polycystic ovary syndrome (PCOS), one of the most common endocrinopathies in premenopausal women, which has been redefined as a reproductive and metabolic disorder after the recognition of the important role of insulin resistance in the pathophysiology of the syndrome. Obesity, in particular central adiposity and insulin resistance are considered as the main factors related to NAFLD in PCOS. Although the natural history of NAFLD remains unclear and hepatic steatosis seems to be a relatively benign condition in most patients, limited data imply that advanced stage of liver disease is possibly more frequent in obese PCOS patients with NAFLD [Vassilatou E, World J Gastroenterol. Jul. 14, 2014; 20(26): 8351-8363].

Since current treatment strategies for PCOS have limited efficacy and marked side effects, and further there are no consistent guidelines for their uses, a therapeutic option which can cure the underlying pathophysiology related to hypothalamic pituitary gonadal (HPG) axis, correct the LH pulsatility, and treat the symptoms of metabolic disturbances without significant side effects is needed. Thus, there exists a need for newer therapies for the treatment of polycystic ovarian syndrome (PCOS).

WO03009841 discloses compounds of the following general formula (A)

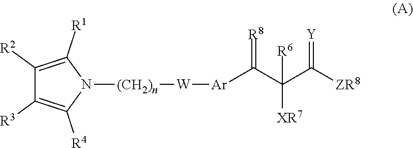

These compounds are reported to be hypolipidaemic agents. This document also discloses sodium and calcium salts of some of the compounds disclosed therein.

WO2012104869 discloses Saroglitazar (I) and its Magnesium salt being effective in the treatment of lipohypertrophy, lipoatrophy and metabolic abnormalities in HIV patients.

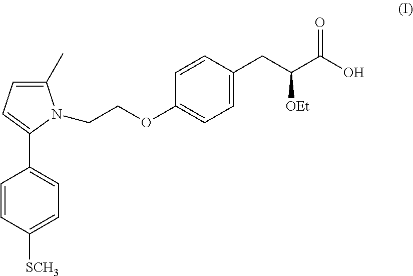 [SMILES: CCO[C@@H](Cc1ccc(OCCn2c(C)ccc2-c2ccc(SC)cc2)cc1)C(=O)O]

WO2014174524 discloses the use of Saroglitazar (I) and its pharmaceutically acceptable salts for the treatment of Non-alcoholic Fatty Liver Diseases (NAFLD) & Nonalcoholic Steatohepatitis (NASH). WO2016181409 discloses the use of Saroglitazar and its pharmaceutically acceptable salts for the treatment of Chylomicronemia. WO2017089979 discloses the use of the compound (I) and its pharmaceutically acceptable salts for the treatment of diabetic nephropathy. WO2017089980 discloses the use of the compound (I) and

its pharmaceutically acceptable salts for the treatment of diabetic retinopathy. Disclosed herein are the use of the compound (I) or its pharmaceutically acceptable salts for the treatment of polycystic ovarian syndrome (PCOS).

### OBJECTIVE OF THE INVENTION

In one embodiment, the present invention discloses a pharmaceutical composition comprising the compound of the formula (I) or its pharmaceutically acceptable salts for treatment of polycystic ovarian syndrome (PCOS) in patients in need of such treatment.

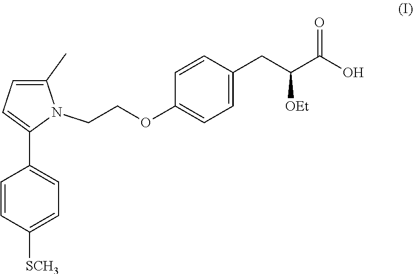 [SMILES: CCO[C@@H](Cc1ccc(OCCn2c(C)ccc2-c2ccc(SC)cc2)cc1)C(=O)O]

In one embodiment, the present invention discloses a pharmaceutical composition comprising the compound of the formula (Ia) for treatment of polycystic ovarian syndrome (PCOS) in patients in need of such treatment.

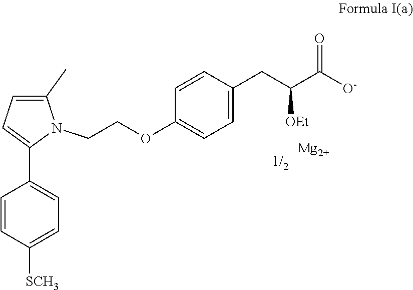

In another embodiment the present invention provides a method of treating polycystic ovarian syndrome (PCOS) in a subject, comprising administering to the subject an effective amount of a compound according to formula (I), or a pharmaceutically acceptable salts thereof as a suitable pharmaceutically acceptable composition.

In another embodiment the present invention provides a method of treating polycystic ovarian syndrome (PCOS) in a subject, comprising administering to the subject an effective amount of a compound according to formula (Ia), as a suitable pharmaceutically acceptable composition.

In a further embodiment is provided use of compound of formula (I) or its pharmaceutically acceptable salts for the treatment of polycystic ovarian syndrome (PCOS).

In a further embodiment is provided use of compound of formula (Ia) for the treatment of polycystic ovarian syndrome (PCOS).

In an embodiment, the present invention discloses a pharmaceutical composition comprising the compound of the formula (I) or its pharmaceutically acceptable salts for treatment of polycystic ovarian syndrome (PCOS) in patients with NAFLD.

In an embodiment, the present invention discloses a pharmaceutical composition comprising the compound of the formula (Ia) for treatment of polycystic ovarian syndrome (PCOS) in patients with NAFLD.

In another embodiment the present invention provides a method of treating polycystic ovarian syndrome (PCOS) in patients with NAFLD, comprising administering to the subject an effective amount of a compound according to formula (I), or a pharmaceutically acceptable salts thereof as a suitable pharmaceutically acceptable composition.

In another embodiment the present invention provides a method of treating polycystic ovarian syndrome (PCOS) in patients with NAFLD, comprising administering to the subject an effective amount of a compound according to formula (Ia), as a suitable pharmaceutically acceptable composition

In a further embodiment is provided use of compound of formula (I) or its pharmaceutically acceptable salts for the treatment of polycystic ovarian syndrome (PCOS) in patients with NAFLD.

In a further embodiment is provided use of compound of formula (Ia) for the treatment of polycystic ovarian syndrome (PCOS) in patients with NAFLD.

The above and other embodiments of the present invention are disclosed further hereinafter.

### DESCRIPTION OF THE FIGURES

FIG. 1: AUC glucose during insulin tolerance test after 4 weeks of treatment with Compound of formula (Ia), Metformin, and combination of Compound of formula (Ia) and Metformin in letrozole-induced PCOS in female Zucker fa/fa rats.

FIG. 2: Serum LH level after 4 weeks of treatment with Compound of formula (Ia), Metformin, and combination of Compound of formula (Ia) and Metformin in letrozole-induced PCOS in female Zucker fa/fa rats.

FIG. 3: Serum FSH level after 4 weeks of treatment with Compound of formula (Ia)), Metformin, and combination of Compound of formula (Ia) and Metformin in letrozole-induced PCOS in female Zucker fa/fa rats.

FIG. 4: Serum testosterone level after 4 weeks of treatment with Compound of formula (Ia), Metformin, and combination of Compound of formula (Ia) and Metformin in letrozole-induced PCOS in female Zucker fa/fa rats.

FIG. 5: LH/FSH ratio after 4 weeks of treatment with Compound of formula (Ia), Metformin, and combination of Compound of formula (Ia) and Metformin in letrozole-induced PCOS in female Zucker fa/fa rats.

FIG. 6: Ovaries weight after 4 weeks of treatment with Compound of formula (Ia), Metformin, and combination of Compound of formula (Ia) and Metformin in letrozole-induced PCOS in female Zucker fa/fa rats.

FIG. 7: Number of cystic follicles after 4 weeks of Compound of formula (Ia) treatment. Normal control refers to the animals not treated with Letrozole (i.e., non PCOS female Wistar Rats).

### DEFINITIONS AND ABBREVIATIONS

As used above, and throughout this disclosure, the following terms, unless otherwise indicated, shall be understood to have the following meanings:

“Patient” includes both human and animals. “Mammal” means humans and other mammalian animals.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein “treating” includes achieving, partially or substantially, one or more of the following results: partially or totally reducing the extent of the disease, disorder or syndrome (e.g., reducing fat deposits, increasing insulin activity/sensitivity, reducing weight); ameliorating or improving a clinical symptom or indicator associated with the disorder; delaying, inhibiting or preventing the progression of the disease, disorder or syndrome; or partially or totally delaying, inhibiting or preventing the onset or development of disorder.

### DETAILED DESCRIPTION OF THE INVENTION

In one aspect the present invention describes a pharmaceutical composition of compound of formula (I) or its suitable pharmaceutically acceptable salts for treatment of polycystic ovarian syndrome (PCOS).

The pharmaceutical composition comprises compound of formula (I) or its pharmaceutically acceptable salts along with suitable pharmaceutically acceptable excipients and the method comprises administering to a subject in need thereof an effective amount of the compound according to Formula (I), or a pharmaceutically acceptable salt thereof.

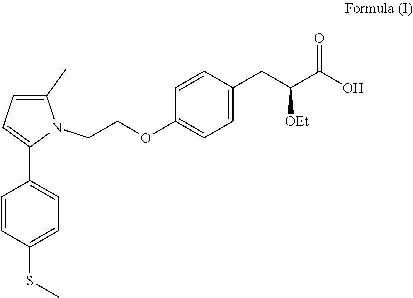 [SMILES: CCO[C@@H](Cc1ccc(OCCn2c(C)ccc2-c2ccc(SC)cc2)cc1)C(=O)O]

In a preferred embodiment the pharmaceutically acceptable salts are selected from metal cations. In a further preferred embodiment, the pharmaceutically acceptable salts are selected from Na⁺, K⁺, Ca⁺², Mg⁺² and the like. In a particularly preferred embodiment, the compound is the Magnesium salt having formula I(a)

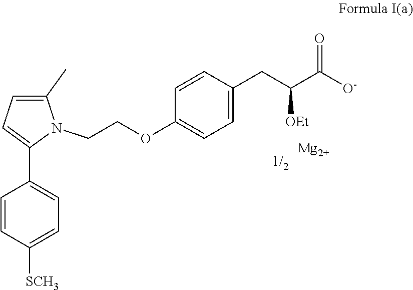

The compound of formula (I) is commercially known as Saroglitazar. This compound (Saroglitazar) is dosed to patients in need thereof as its Magnesium salt (Ia) for the treatment of one or more of the diseases described above.

The compounds of formula (I) can form salts which are also within the scope of this invention. Reference to a compound of formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formula (I) contain both a basic moiety, such as, but not limited to a pyridine or imidazole, and an acidic moiety, such as, but not limited to a carboxylic acid, zwitterions (“inner salts”) may be formed and are also included within the term “salt(s)” as used herein.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates.

One or more compounds of the invention may optionally be converted to a solvate. Preparation of solvates is generally known. Thus, for example, M. Caira et al, J. Pharmaceutical Sci, 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Common., 603-604 (2001).

Pharmaceutically acceptable (i.e., non-toxic, physiologically acceptable) salts are preferred, although other salts are also useful. Salts of the compounds of the formula (I) may be formed, for example, by reacting a compound of formula (I) with an amount of acid or base, such as an equivalent amount, in a medium such as one in which the salt precipitates or in an aqueous medium followed by lyophilization.

Polymorphic forms of the compounds of formula (I), and of the salts, solvates, esters and prodrugs of the compounds of formula (I) are intended to be included in the present invention. The compound of formula (I), its pharmaceutically acceptable salts and their pharmaceutical compositions were prepared as per the processes disclosed in the prior art such as those mentioned elsewhere in the specification.

As used herein, the term “composition” is intended to encompass a product comprising the specified excipients in the specified amounts, as well as any product which results, directly or indirectly, from combination of the specified excipients in the specified amounts.

In the embodiments the present invention provides a suitable pharmaceutical composition of compounds of formula (I) or its pharmaceutically acceptable salts, which comprises one or more pharmaceutical excipients, antioxidants and chelating agents, wherein the pH of the composition is above 6, preferably in the range from about pH 6 to pH of about 10.

In such embodiments the pharmaceutical composition of the present invention essentially comprises of
compound of formula (Ia),
suitable alkalinizers or suitable pH modifying agents which maintain the pH of the formulation above 7, and optionally
a suitable stabilizer (antioxidants and chelating agents);
and one or more other pharmaceutically acceptable excipients.

In an embodiment suitable stabilizers may be selected from the classes of antioxidants or chelating agents.

In an embodiment the pharmaceutical excipients according to the present invention can be selected from solubilizers, diluents, fillers, disintegrants, binder, lubricants, glidants, wetting agents, solvents and the like as is known in the art.

In embodiment suitable additives are selected from sodium benzoate, sodium hydroxide, sodium sulfite and sodium carbonate.

In an embodiment antioxidants used according to the present invention include, but are not limited to citric acid, alpha tocopherol, sodium sulphite, sodium metabisulphite, butylated hydroxy anisole (BHA), BHT (2,6-di-tert-butyl-4-methylphenol), monothioglycerol, Vitamin C (ascorbic acid), and propyl gallate and combinations thereof and other similar material known to those of ordinary skilled in the art.

Alkalinizers or suitable pH modifying agents which maintain the pH of the formulation above 7 used according to the present invention include, but are not limited to attapulgite, bentonite, calcium carbonate, calcium phosphate, calcium sulphate, mono ethanolamine, tri ethanolamine, potassium bicarbonate, potassium citrate, potassium hydroxide, sodium benzoate, sodium hydroxide, sodium sulfite, sodium bicarbonate, sodium carbonate, Disodium Hydrogen phosphate, mono basic potassium phosphate, Dicalcium phosphate, meglumine, light or heavy magnesium oxide and other similar excipients and their suitable combinations and other materials known to those of ordinary skill in the art.

Chelating agent used according to the present invention include, but are not limited to Disodium EDTA, citric acid and or its salts, maleic acid, chlorambutol, chlorhexidine or its salts, chlorocresol, combinations thereof and other similar material known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methyl cellulose, povidone and pregelatinized starch, combinations thereof and other similar material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC F68, PLURONIC F127), collagen, albumin, celluloses in non-aqueous solvents, and the like or their suitable combinations. Other binders which may be included may be, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art. As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

In an embodiment, the term “lubricant” is intended to mean substances used in tablet formulations to reduce friction during tablet compression. Such compounds include, by way of example and without limitation, calcium stearate, magnesium stearate, mineral oil, stearic acid, zinc stearate, suitable combinations thereof and other such materials known to those of ordinary skill in the art.

In an embodiment, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), Croscarmellose Sodium (acdisol), alginates, sodium starch glycolate, gums such as agar, guar, locust bean, karaya, pectin, tragacanth, combinations thereof and other such materials known to those of ordinary skill in the art.

In an embodiment, the term “wetting agent” is intended to mean a compound used to aid in attaining intimate contact between solid particles and liquids. Exemplary wetting agents include, by way of example and without limitation, poloxamers, gelatin, casein, Glycerol mono-oleate, lecithin (phosphatides), gum acacia, cholesterol, tragacanth, stearic acid, benzalkonium chloride, calcium stearate, glycerol monostearate, cetostearyl alcohol, sodium lauryl sulphate, sodium dodecyl sulfate, salts of bile acids (taurocholate, glycocholate, cholate, deoxycholate, etc.), cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN), polyethylene glycols, polyoxyethylene stearates colloidal silicon dioxide, phosphates, sodium dodecylsulfate, carboxymethylcellulose calcium, carboxy methylcellulosesodium, methyl cellulose, hydroxyethylcellulose, hydroxylpropylcellulose, hydroxy propyl methyl cellulose phthalate, noncrystalline cellulose, magnesium aluminum silicate, triethanolamine, polyvinyl alcohol, and poly vinyl pyrrolidone (PVP) & their suitable combinations and other such materials known to those of ordinary skill in the art. Tyloxapol (a nonionic liquid polymer of the alkyl aryl polyether alcohol type, also known as superinone or triton) is another useful wetting agent which may be used. The stable pharmaceutical composition according to the present invention may be in the form of tablet or capsule or a powder or a suspension in a liquid or an aerosol formulation or solutions, preferably in the form of tablet or capsule.

Stable pharmaceutical composition of compounds of formula (I) or its pharmaceutically acceptable salts may have been prepared as process provided in WO2015011730.

The stable pharmaceutical composition may be made by direct compression, wet granulation or dry granulation methods by techniques known to persons skilled in the art. Thus, for example,

In wet granulation process, the drug is mixed with one or more pharmaceutical excipients and granulated with suitable binding solution as described earlier, to form wet granules, the wet granules are dried and optionally sieved. The dried granules are mixed with one or more suitable excipients from those described elsewhere and then compressed into tablets or filled into capsules.

In direct compression process, the drug is mixed with all the pharmaceutical excipients required and then is either compressed into tablets or filled in capsules.

In dry granulation process the drug is mixed with one or more pharmaceutical excipients and compressed into slugs and these slugs are passed through required sieve. The sieved granules are mixed with one or more suitable excipients from those described elsewhere and then compressed into tablets or filled into capsules.

One or more solvents used in the formulation are selected from acetone, chloroform, dichloromethane, ethyl alcohol, ethyl acetate, methyl alcohol, isopropyl alcohol and combinations thereof and other such materials known to those of ordinary skill in the art.

In one of the embodiment, present invention discloses the prepared pharmaceutical composition of compound of formula (I) is to be administered orally.

In an embodiment, the compound of formula (I) or pharmaceutical compositions containing the compound of formula (I) is given to a subject in need thereof at a dose of about 0.5 mg to 10 mg In a preferred embodiment, the compound of formula (I) or pharmaceutical compositions containing the compound of formula (I) is given to a subject in need thereof at a dose of about 0.5 mg to 10 mg, such doses may include doses of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg as well as any intermittent dosages which are within the scope of a person of ordinary skill.

In another embodiment, the compound of formula (I) or pharmaceutical composition is for administration to a human subject.

In an embodiment, the compounds of formula (I) or its pharmaceutically acceptable salts can be used alone or in combination with an additional therapeutic agent.

In an embodiment, the compounds of formula (Ia) in combination with an additional therapeutic agent.

An additional therapeutic agent is selected from the drugs that are metformin, SGLT2 inhibitor or DPP-IV inhibitors such as, vildagliptin dapagliflozine etc.

In one of the embodiment, pharmaceutical composition comprising compound of formula (I) and metformin as an additional therapeutic agent for use in treating or ameliorating polycystic ovarian syndrome (PCOS).

In one of the embodiment, a pharmaceutical composition comprising compound of formula (Ia) and metformin as an additional therapeutic agent for use in treating or ameliorating polycystic ovarian syndrome (PCOS). The individual therapeutic agents can be given either separately, sequentially or may be co-formulated into a single pharmaceutical composition with suitable excipients as is well within the scope of a skilled person.

In an embodiment, the compound of formula (I) or its pharmaceutical composition are also useful in treatment or amelioration of polycystic ovarian syndrome in a subject that are diagnosed with Nonalcoholic fatty acid liver disease (NAFLD).

In another preferred embodiment, the pharmaceutical composition comprising compound of formula (Ia) are also useful in treatment or amelioration of polycystic ovarian syndrome in a subject that are diagnosed with Nonalcoholic fatty acid liver disease (NAFLD).

In one of the specific embodiment, the pharmaceutical composition comprising compound of formula (Ia) and metformin as an additional therapeutic agent for use in treating or ameliorating polycystic ovarian syndrome (PCOS) in the subject that are diagnosed with Nonalcoholic fatty acid liver disease (NAFLD).

The present invention also provides the method of treating polycystic ovarian syndrome (PCOS) by administering effective amount of compound of formula (I) or its pharmaceutically acceptable salt.

In an embodiment, present invention also discloses the method of treating polycystic ovarian syndrome (PCOS) by administering effective amount of pharmaceutically acceptable salts of compound of formula (I). Pharmaceutically acceptable salts include metal cation salts of compound of formula (I) such as sodium, potassium, calcium, magnesium, etc. salts. In a preferred embodiment, present invention discloses method of treating polycystic ovarian syndrome (PCOS) by administering effective amount of magnesium salt of compound of formula (I) which is also known as Saroglitazar magnesium (formula-(Ia)).

According to present invention, in the method of treating polycystic ovarian syndrome, compound of formula (I) or its pharmaceutically acceptable salts are administered in the form of pharmaceutical formulation.

In a particular embodiment present invention provides a pharmaceutical formulation comprising compound of formula (I) or its pharmaceutically acceptable salts and also comprise suitable excipients such as additives, stabilizers, glidant, solubilizers, diluents, fillers, disintegrants, binder, lubricants, solvents, wetting agents, etc.

The method of treating polycystic ovarian syndrome can be characterized on the basis of route of administration.

For example, in certain embodiments, a pharmaceutical formulation of compound of formula (I) is administered orally when used in method of treating polycystic ovarian syndrome.

The method of treating polycystic ovarian syndrome can be characterized on the basis of dosing amount of compound of formula (I), its pharmaceutically acceptable salts or the pharmaceutical formulation comprising compound of formula (I). In few embodiments, compound of formula (I) or preferably compound of formula (Ia) is administered at a dose in range of 0.5 mg to 10 mg, such doses may include doses of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg as well as any intermittent dosages which are within the scope of a person of ordinary skill. In some of the preferred embodiments, compound of formula (I) is administered at a dose in range of 0.5 mg to 5.0 mg. In certain embodiments, magnesium salt of compound of formula (Ia) is administered at a dose of 4.0 mg.

The method of treating polycystic ovarian syndrome can be characterized by the subject which treatment has been treated. In few embodiments, the subject is human, preferably women.

In another embodiment, present invention provides a method of treating polycystic ovarian syndrome (PCOS) wherein pharmaceutical formulation of compounds of formula (I) or its pharmaceutically acceptable salts is used alone or in combination with an additional therapeutic agent. An additional therapeutic agent is selected from the drugs that are metformin, SGLT2 inhibitor or DPP-IV inhibitors such as Vildagliptin, teneligliptin, sitagliptin and the like or SGLT2 inhibitors such as dapagliflozin, canagliflozin, empagliflozin and the like, etc.

In certain embodiment present invention provides a method of treating polycystic ovarian syndrome (PCOS), wherein pharmaceutical formulation of compounds of formula (Ia) is used in combination with metformin as an additional therapeutic agent.

The method of treating polycystic ovarian syndrome can also be characterized on the bases of dosing schedule and duration of the treatment. In few embodiments, compound of formula (I) or its pharmaceutically acceptable salt administered once in a day, twice in a day or as per the requirement of the subject/patient. In few embodiments, treatment of polycystic ovarian syndrome has been carried out for at least 5 days to 9 months as per the requirement of subject/patient.

The method of treating polycystic ovarian syndrome can be characterized on the basis of change in level of LH, FSH and testosterone in the body. It can also be characterized on the base of change in LH to FSH ratio.

Patients with PCOS have their LH and FSH levels in the range of 1.5-8 ng/ml, in which the ratio of LH to FSH ratio is more than one. Due to this comparative elevation in LH levels, the ovulation process is disrupted and cysts may occur. In certain embodiments, treatment with compound of formula (I) may decrease this ratio to normal, i.e., below one, especially if combined with metformin. Another important factor in PCOS is elevated testosterone levels in blood. Total testosterone is 6.0-86 ng/dl. In certain embodiments testosterone level is reduced by treatment with compound of formula (I). This decrease will be further enhanced, if combined with metformin.

The present invention also provides the method of treating polycystic ovarian syndrome (PCOS) in a subject diagnosed with NAFLD, by administering effective amount of compound of formula (I) or its pharmaceutically acceptable salt.

In an embodiment, present invention also discloses the method of treating polycystic ovarian syndrome (PCOS) in a subject diagnosed with NAFLD, by administering effective amount of pharmaceutically acceptable salts of compound of formula (I). Pharmaceutically acceptable salts include metal cation salts of compound of formula (I) such as sodium, potassium, calcium, magnesium, etc. salts. In a preferred embodiment, present invention discloses method of treating polycystic ovarian syndrome (PCOS) in a subject diagnosed with NAFLD by administering effective amount of magnesium salt of compound of formula (Ia) which is also known as Saroglitazar magnesium.

According to present invention, in the method of treating polycystic ovarian syndrome in a subject diagnosed with NAFLD, compound of formula (I) or its pharmaceutically acceptable salts are administered in the form of pharmaceutical formulation.

In a particular embodiment present invention provides a pharmaceutical formulation comprising compound of formula (I) or its pharmaceutically acceptable salts and also comprise suitable excipients such as additives, stabilizers, glidant, solubilizers, diluents, fillers, disintegrants, binder, lubricants, solvents, wetting agents, etc.

The method of treating polycystic ovarian syndrome in a subject diagnosed with NAFLD can be characterized on the basis of route of administration. For example, in certain embodiments, a pharmaceutical formulation of compound of formula (I) is administered orally when used in method of treating polycystic ovarian syndrome.

The method of treating polycystic ovarian syndrome in a subject diagnosed with NAFLD can be characterized on the basis of dosing amount of compound of formula (I), its pharmaceutically acceptable salts or the pharmaceutical formulation comprising compound of formula (I). In few embodiments, compound of formula (I) is administered at a dose in range of 0.5 mg to 10 mg. In some of the preferred embodiments, compound of formula (I) is administered at a dose in range of 0.5 mg to 10.0 mg such doses may include doses of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg as well as any intermittent dosages which are within the scope of a person of ordinary skill. In certain embodiments, magnesium salt of compound of formula (Ia) is administered at a dose of 4.0 mg.

The method of treating polycystic ovarian syndrome in a subject diagnosed with NAFLD can be characterized by the subject which treatment has been treated. In few embodiments, the subject is human, preferably women.

In another embodiment present invention provides a method of treating polycystic ovarian syndrome (PCOS) in a subject diagnosed with NAFLD, wherein pharmaceutical formulation of compounds of formula (I) or its pharmaceutically acceptable salts is used alone or in combination with an additional therapeutic agent. An additional therapeutic agent is selected from the drugs that are metformin, SGLT2 inhibitor or DPP-IV inhibitors such as Vildagliptin, teneligliptin, sitagliptin and the like or SGLT2 inhibitors such as dapagliflozin, canagliflozin, empagliflozin and the like etc. In certain embodiment present invention provides a method of treating polycystic ovarian syndrome (PCOS) in a subject diagnosed with NAFLD, wherein pharmaceutical formulation of compounds of formula (Ia) is used in combination with metformin as an additional therapeutic agent.

The method of treating polycystic ovarian syndrome in a subject diagnosed with NAFLD can also be characterized on the bases of dosing schedule and duration of the treatment. In few embodiments, compound of formula (I) or its pharmaceutically acceptable salt administered once in a day, twice in a day or as per the requirement of the subject/patient. In few embodiments, treatment of polycystic ovarian syndrome has been carried out for at least 5 days to 9 months as per the requirement of subject/patient.

A skilled person is aware how to decide dosage amount of drug, dosage schedule and duration of treatment in a subject/patient diagnosed with NAFLD on the basis of patient profile, the severity of disease, the presence of secondary medicines and the like.

The method of treating polycystic ovarian syndrome in a subject diagnosed with NAFLD can be characterized on the basis of change in level of LH, FSH and testosterone in the body. It can also be characterized on the base of change in LH to FSH ratio.

Patients with PCOS have their LH and FSH levels in the range of 1.5-8 ng/ml, in which the ratio of LH to FSH ratio is more than one. Due to this comparative elevation in LH levels, the ovulation process is disrupted and cysts may occur. In certain embodiments, treatment with compound of formula (I) may decrease this ratio to normal, i.e., below one, especially if combined with metformin. Another important factor in PCOS is elevated testosterone levels in blood. Total testosterone is 6.0-86 ng/dl. In certain embodiments, testosterone level is reduced by treatment with compound of formula (I). This decrease will be further enhanced, if combined with metformin.

The present invention is further exemplified by the following non-limiting examples.

### EXAMPLES

### Example 1 Composition of Compound of Formula I(a)

| Ingredient | % w/w |
|---|---|
| Compound (Ia) | 3.08 |
| Light magnesium oxide | 6.15 |
| Lactose Anhydrous | 23.07 |
| Microcrystalline cellulose | 50.00 |
| Acdisol | 9.23 |
| Povidone K-30 | 3.85 |
| Purified Talc | 1.54 |
| Aerosil | 1.54 |
| Magnesium Stearate | 1.54 |

The efficacy of the compound in the treatment of PCOS is evaluated in vivo as follows:

A. Study Design & Protocol:

Clinically, PCOS is characterized by irregular or absent menstrual cycles, abdominal obesity, and signs of androgen excess (hyperandrogenism) which include acne or seborrhea and insulin resistance. Lipid imbalance, oxidative stress, insulin resistance, and genetic predisposition also add to the pathogenesis of PCOS. Letrozole, a non-steroidal aromatase inhibitor induces PCOS in rats by blocking conversion of testosterone and androstenedione to estradiol and estrone respectively and simulates PCOS like condition by causing hormonal imbalance, circulating hyperandrogenism and intra ovarian androgen excess leading to appearance of polycystic ovary Follicular atresia and abnormal follicular development is observed due to induced elevation of androgen levels inside the ovary. Letrozole also causes insulin resistance and hyperlipidemia.

Zucker fa/fa rats are used in a wide variety of research areas including cardiovascular biology, developmental biology, diabetes and obesity research. Zucker fa/fa rats breed well, are long-lived, and have a low susceptibility to tumors. Zucker fa/fa rats can develop obesity, hyperinsulinemia and PCOS. Therefore, this model proves to be suitable for predicting the likely therapeutic benefit of novel treatment for PCOS in humans.

### Example 2

### 1. Objective

The objective of this study was to assess the effect of Compound of formula I(a) in
Letrozole-
   induce polycystic ovary syndrome (PCOS) in female Zucker fa/fa rats.

### 2. Materials

### 2.1 Chemicals

Carboxy methyl cellulose (Sigma; Catalogue No. 419338), Tween80 (Sigma; Catalogue No P1754), Poly ethylene glycol 400 (Aldrich; Catalogue No. P3265), Sodium chloride (Merck; Catalogue No. ME0M601226), Letrozole (TCI chemicals, India Catalogue No. L0248), Metformin (Hydrochloride salt, EXE Pharmaceuticals, Catalogue No. MFH160497AFP), ISOFLURANE USP, (Raman & weil India Pvt LTD, Batch No.-IF-17002), Insulin (Torrent pharma LTD; Batch No. B-70321), Glucose (Merck; Batch No MK7M573059), Glucose strip (Lifescan; Lot No 4277298), LH (Neo scientific; Catalogue No RF0028), FSH (Neo scientific; Catalogue No RF0008) and testosterone (Crystal chem; Catalogue No 80550) estimation kits were used during this study.

### 2.2 Instruments

Sartorius semi-micro balance (model No.-ME235P) was used for weighing the compound. Sartorius balance (model No.-LA4200S) was used for weighing the animals. One Touch Ultra glucometer, Life Scan Inc., USA was used for glucose estimation and Synergy HT multi detection micro plate reader (BioTek instruments) was used for plate reading.

### 3 Test Item Details

| Test Item | | Chromatographic | |
|---|---|---|---|
| Name | Batch | Purity (%) by HPLC | Storage conditions |
| Compound | SRC1ASB04D | 101% w/w | Store the material in tightly closed |
| of formula I(a) | | | container at temperature 5 ± 3° C. |
| Metformin | MFH160497AFP | 100.1% w/w | Store the material in tightly closed |
| (Hydrochloride | | | container at temperature 25 ± 2° C. |
| salt) | | | |

2.4 Vehicle—Tween80 (0.5% v/v), PEG 400 (0.5% v/v) in (0.5% w/v) carboxy methyl cellulose in deionized water.

### 2.5 Formulation

Letrozole were suspended in Tween80 (0.5% v/v), PEG 400 (0.5% v/v) in (0.5% w/v) carboxy methyl cellulose in deionized water. Compound of Formula (Ia) was also formulated in the similar suspension. The formulations were prepared and administered at 2 mL/kg.

### 2.6 Test System

### Species: Rats

Strain: Zucker fa/fa

### Sex: Female

Source: Animal breeding Facility of Zydus Research Centre, registered under Rule 5(a) for the “Breeding and Experiments on Animals (control and supervision) rules 1998 (Registration no. 77/1999/CPCSEA).
Body weight range: 348.6 to 492.6 g at initiation of study
Age at initiation: 13-15 weeks old at initiation of study
Identification system: Animals were identified by ear punch mark. Individual cage cards were affixed to each cage that displayed the cage No., species, strain, sex, No. of animals, date of initiation, date of termination, study number and study scientist.

### 3. Test Conditions

### Animal Housing

Animals were housed one or two per cage in cleaned polypropylene cages. Cages were filled with autoclaved corn-cob as the bedding material.

### Drinking Water

Purified water was provided using water bottles ad libitum.

### Basal Diet

Teklad Global 18% Protein Rodent Diet (Envigo; Catalogue No 2018) was offered ad libitum during acclimatization.

### 4. Methods and Experimental Design

Female Zucker fa/fa rats (6-8 weeks) were issued from animal house of Zydus research Centre and quarantined for 7 weeks. At the age of 13-15 weeks all animals were administered Letrozole (1 mg/kg/2 ml) orally for 4 weeks to induce PCOS.

After 4 weeks of Letrozole, all animals with confirmed PCOS on basis of body weight and estrous cycle were randomized. After randomization, animals were assigned to the oral treatment of either Vehicle control (n=6), Compound of Formula (Ia) (4 mg/kg) treatment (n=6), Metformin (200 mg/kg) (n=6), or Combination of Compound of Formula (Ia) (4 mg/kg) and Metformin (200 mg/kg) (n=6). Letrozole (1 mg/kg/2 mL) treatment was continued.

After 4 weeks of compound of formula (Ia) treatment, Insulin was subcutaneously injected at 1.5 U/kg regular insulin and 2 g/kg glucose load was administered orally. Blood was collected 40 min following injection. Serum samples were analyzed for LH, FSH and testosterone using ELISA kit according to manufacturer's instructions.

Continuing compound of formula (Ia) treatment for next two days, insulin tolerance test was performed after 6 h fast. Insulin 1.51 U/kg was administered intraperitoneally at 0 min. Whole blood glucose was estimated using One-touch ultra-blood glucose meter. Blood glucose levels were then estimated at 0, 15, 30, 60 and 120 min corresponding to the insulin injection.

In similar manner as described above, effects of metformin and effect of the combination of compound of formula (Ia) and metformin were also measured.

At the end of study, animals were sacrificed by cervical dislocation and ovaries were collected and weighed.

### 5. Calculations and Statistical Evaluation

Data represents Mean±SEM, statistically significant difference was determined by an unpaired test using Graph Pad Prism software version 7.03. P<0.05 is considered as statistically significant.

### 6. Results

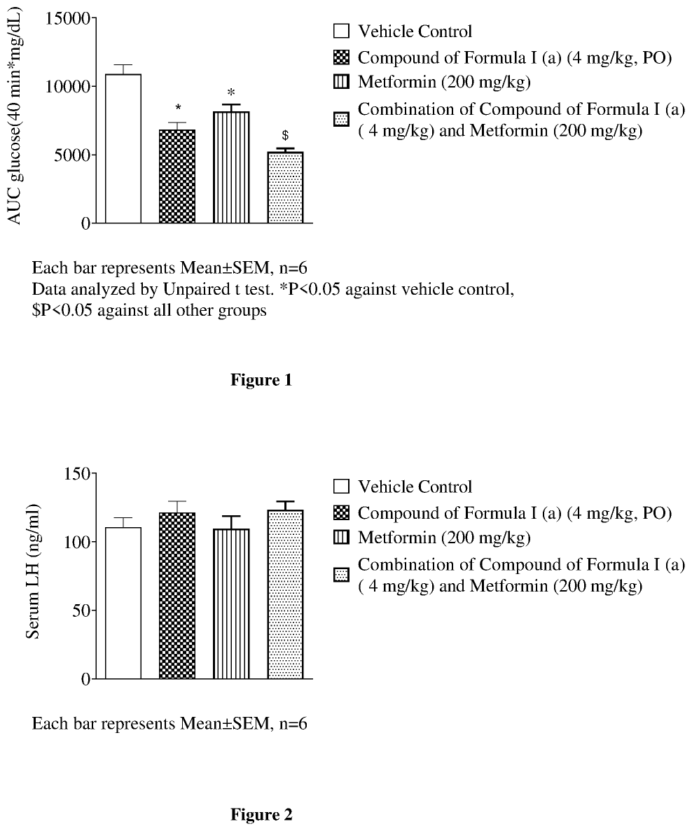

TABLE 1
AUC glucose during insulin tolerance test after 4 weeks of treatment with compound
of formula (Ia), Metformin, and combination of Compound of Formula (Ia) and Metformin in
letrozole-induced PCOS in female Zucker fa/fa rats (FIG. 1)

| | AUC glucose (120 | % Change Vs Vehicle | Statistical |
|---|---|---|---|
| Treatment group | min*mg/dL) | Control | significance |
| Vehicle Control | 10890.2 ± 677.5 | | |
| Compound of Formula (Ia) | 6819.0 ± 535.8 | −37.4 ± 4.9 | P <0.05 |
| (4 mg/kg) | | | |
| Metformin (200 mg/kg) | 8172.5 ± 500.1 | −24.9 ± 4.8 | P <0.05 |
| Combination of Compound of | 5215.7 ± 246.5 | −47.6 ± 3.1 | P <0.05, |
| Formula (Ia)) (4 mg/kg) and | | | when compared |
| Metformin (200 mg/kg) | | | to all other |
| | | | groups |

TABLE 2
Serum LH level after 4 weeks of treatment with compound of formula (Ia),
Metformin, and combination of Compound of Formula (Ia) and Metformin in letrozole-induced
PCOS in female Zucker fa/fa rats. (FIG. 2)

| | | % Change Vs Vehicle | Statistical |
|---|---|---|---|
| Treatment group | Serum LH (ng/mL) | Control | significance |
| Vehicle Control | 110.5 ± 7.1 | | |
| Compound of Formula (Ia) | 121.4 ± 8.2 | 9.9 ± 7.4 | NS |
| (4 mg/kg) | | | |
| Metformin (200 mg/kg) | 109.6 ± 9.2 | 0.81 ± 3.1 | NS |
| Combination of Compound of | | | |
| Formula (Ia) (4 mg/kg) and | 123.3 ± 6.2 | 11.6 ± 8.1 | NS |
| Metformin (200 mg/kg) | | | |

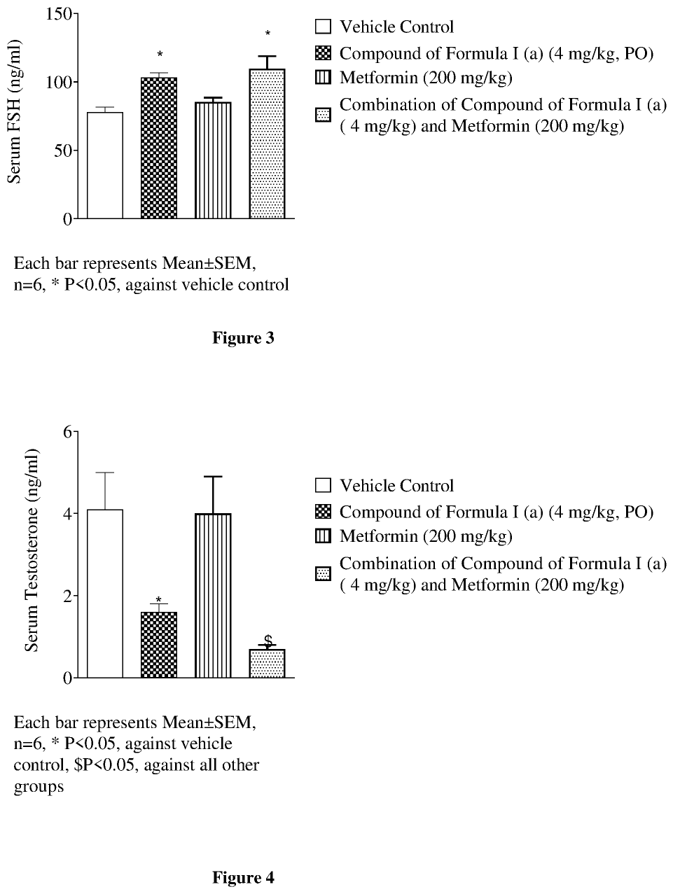

TABLE 3
Serum FSH level after 4 weeks of treatment with compound of formula (Ia),
Metformin, and combination of Compound of Formula (Ia) and Metformin in letrozole-induced
PCOS in female Zucker fa/fa rats. (FIG. 3)

| | | % Change Vs | Statistical |
|---|---|---|---|
| Treatment group | Serum FSH (ng/mL) | Vehicle Control | significance |
| Vehicle Control | 77.9 ± 3.8 | | |
| Compound of Formula (Ia) | 103.1 ± 3.5 | 32.3 ± 4.5 | P <0.05 |
| (4 mg/kg) | | | |
| Metformin (200 mg/kg) | 85.3 ± 3.2 | 4.4 ± 3.6 | NS |
| Combination of Compound of | | | |
| Formula (Ia) (4 mg/kg) and | 109.6 ± 9.2 | 40.7 ± 5.1 | P <0.05 |
| Metformin (200 mg/kg) | | | |

TABLE 4
Serum testosterone level after 4 weeks of treatment with compound of formula (Ia),
Metformin, and combination of compound of Formula (Ia) and Metformin in letrozole-induced
PCOS in female Zucker fa/fa rats. (FIG. 4)

| | Serum testosterone | % Change Vs Vehicle | Statistical |
|---|---|---|---|
| Treatment group | (ng/mL) | Control | significance |
| Vehicle Control | 4.1 ± 0.9 | | |
| Compound of Formula (Ia) | 1.6 ± 0.2 | −61.8 ± 4.7 | P <0.05 |
| (4 mg/kg) | | | |
| Metformin (200 mg/kg) | 4.0 ± 0.9 | −2.4 ± 4.1 | NS |
| Combination of Compound of | 0.7 ± 0.1 | −82.9 ± 4.2 | P <0.05, when |
| Formula (Ia) (4 mg/kg) and | | | compared to |
| Metformin (200 mg/kg) | | | all other |
| | | | groups |

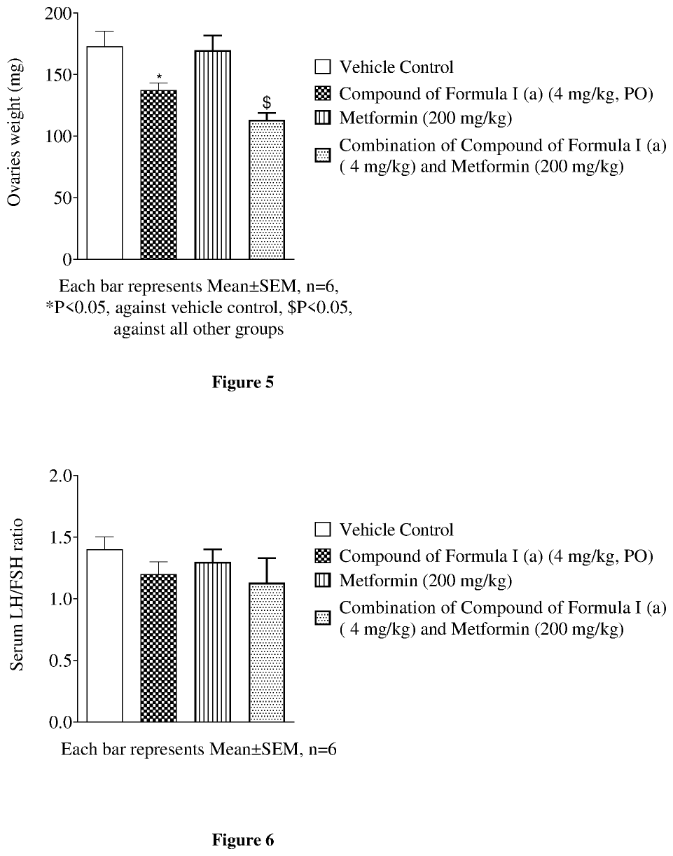

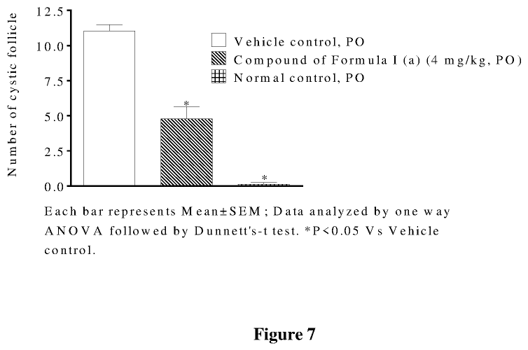

TABLE 5
LH/FSH ratio after 4 weeks of treatment with compound of formula (Ia), Metformin,
and combination of compound of Formula (Ia) and Metformin in letrozole-induced PCOS in
female Zucker fa/fa rats. (FIG. 5)

| | | % Change Vs Vehicle | Statistical |
|---|---|---|---|
| Treatment group | LH/FSH ratio | Control | significance |
| Vehicle Control | 1.4 ± 0.1 | | |
| Compound of Formula (Ia) | 1.2 ± 0.1 | −17.1 ± 6.0 | NS |
| (4 mg/kg) | | | |
| Metformin (200 mg/kg) | 1.3 ± 0.1 | −2.4 ± 4.1 | NS |
| Combination of Compound of | | | |
| Formula (Ia) (4 mg/kg) and | 1.13 ± 0.2 | −19.6 ± 3.6 | NS |
| Metformin (200 mg/kg) | | | |

TABLE 6
Ovaries weight after 4 weeks of treatment with compound of formula (Ia), Metformin,
and combination of compound of Formula (Ia) and Metformin in letrozole-induced PCOS in
female Zucker fa/fa rats. (FIG. 6)

| | | % Change Vs Vehicle | Statistical |
|---|---|---|---|
| Treatment group | Ovaries weight (mg) | Control | significance |
| Vehicle Control | 173.0 ± 12.3 | | |
| Compound of Formula (Ia) | 137.2 ± 6.1 | −20.7 ± 3.5 | P <0.05 |
| (4 mg/kg) | | | |
| Metformin (200 mg/kg) | 169.8 ± 11.9 | −2.4 ± 4.1 | NS |
| Combination of Compound of | 113.3 ± 5.6 | −34.5 ± 2.9 | P <0.05 |
| Formula (Ia) (4 mg/kg) and | | | When |
| Metformin (200 mg/kg) | | | compared to |
| | | | all other |
| | | | groups |

Treatment with compound of formula (Ia) has significantly decreased the occurrence of cystic follicles female Wistar rats in which PCOS was induced using Letrozole. Repeated dose oral treatment with compound of formula (Ia) at 4 mg/kg resulted in significantly improved insulin sensitivity, increased serum FSH, decreased LH/FSH ratio, decreased serum testosterone and normalized ovary weight. When combined with metformin (200 mg/kg), compound of formula (Ia) showed enhanced improvement in insulin sensitivity and decrease in serum testosterone levels in female Zucker fatty rats in which PCOS was induced using Letrozole. In these rats, compound of formula (Ia) and metformin combination also decreased the ovaries weight, indicating an additive effect of compound of formula (Ia) and metformin on reducing symptoms of PCOS.

### Example 3

### 1. Objective

The objective of this study was to assess the effect of compound of formula (Ia) in Letrozole-induced polycystic ovary syndrome (PCOS) in female Wistar rats.

### 2. Materials

### 2.1 Chemicals

Carboxy methyl cellulose (Sigma; Catalogue No. 419338), Tween80 (Sigma; Catalogue No P1754), Poly ethylene glycol 400 (Aldrich; Catalogue No. P3265), Sodium chloride (Merck; Catalogue No. ME0M601226), Letrozole (TCI chemicals, India Catalogue No. L0248), Formalin (Hexa pharmachem), Hematoxylin (Himedia; Catalogue No. GRM9946) and Eosin (Sigma; Catalogue No. HT110316) were the excipients and chemicals used during this study.

### 2.2 Instruments

Sartorius semi-micro balance (model No.-ME235P) was used for weighing the compound. Sartorius balance (model No.-LA4200S) was used for weighing the animals. Leica rotary microtome (model No.-RM2255) was used for sectioning the fixed tissues. Leica light microscope was used for observing tissue sections.

### 2.3 Test Item Details

| | | Chromatographic | |
|---|---|---|---|
| Test Item Name | Batch | Purity (%) by HPLC | Storage conditions |
| Compound of | SRC1ASB04D | 101% w/w | Store in material in tightly closed |
| Formula (Ia) | | | container at temperature 5 ± 3° C. |

2.4 Vehicle—Tween80 (0.5% v/v), PEG 400 (0.5% v/v) in (0.5% w/v) carboxy methyl cellulose in deionized water

### 2.5 Formulation

Letrozole were suspended in Tween80 (0.5% v/v), PEG 400 (0.5% v/v) in (0.5% w/v) carboxy methyl cellulose in deionized water. Compound of Formula (Ia), was also formulated in the similar suspension. The formulations were prepared and administered at 2 mL/kg.

### 2.6 Test System

### Species: Rats

### Strain: Wistar

### Sex: Female

Source: Animal breeding Facility of Zydus Research Centre, registered under Rule 5(a) for the “Breeding and Experiments on Animals (control and supervision) rules 1998 (Registration no. 77/1999/CPCSEA).
Body weight range: 150.7 to 268.3 g at initiation of study
Age at initiation: 11-12 weeks old at initiation of study
Identification system: Animals were identified by ear punch mark. Individual cage cards were affixed to each cage that displayed the cage No., species, strain, sex, No. of animals, date of initiation, date of termination, study number and study scientist.

### 3. Test Conditions

### Animal Housing

Animals were housed four per cage in cleaned polypropylene cages. Cages were filled with autoclaved corn-cob as the bedding material.

### Drinking Water

Purified water was provided using water bottles ad libitum.

### Basal Diet

Teklad Global 18% Protein Rodent Diet (Envigo; Catalogue No 2018) was offered ad libitum during acclimatization.

### 4. Methods and Experimental Design

Sixty female Wistar rats (7-8 weeks) were issued from animal house of Zydus research Centre and quarantined for 4 weeks. At the age of 11-12 weeks all animals were randomized on body weight basis into two treatment groups, namely, vehicle (n=10) as non-PCOS or Letrozole (n=50) for PCOS. Formulated Letrozole (1 mg/kg/2 ml) were administered orally for 4 weeks to induce PCOS.

After 4 weeks of Letrozole, all animals with confirmed PCOS on basis of body weight and estrous cycle were randomized. After randomization, animals were assigned to either Vehicle control or Compound of Formula (Ia) (4 mg/kg, PO) treatment (n=8). Letrozole (1 mg/kg/2 mL) treatment was continued. Rest of the animals was sacrificed.

At the end of treatment, animals were sacrificed by cervical dislocation and ovaries were collected in 10% formalin. Ovaries were processed and embedded in paraffin and subjected to serial sectioning at 3-5 μm using rotary microtome, and sections were transferred on glass slides and stained with haematoxylin-eosin (H and E). The evaluation was done using light microscope and grading of the tissue pathological status was done. Total numbers of cystic follicles were counted on all sections throughout each ovary.

### 5. Calculations and Statistical Evaluation

Data represents mean±SEM, statistically significant difference was determined by a one-way Analysis of Variance (ANOVA) followed by Dunnett's t-test using Graph Pad Prism software version 7.03. P<0.05 is considered as statistically significant.

### 6. Result

TABLE 7
Number of cystic follicles after 4 weeks of
Compound of Formula (Ia) treatment.
Normal control refers to the animals not treated
with Letrozole (i.e., non PCOS female Wistar Rats).

| | Number of cystic | % Change Vs | Statistical |
|---|---|---|---|
| Treatment group | follicle | Vehicle control | significance |
| Vehicle control, PO | 11.04 ± 0.46 | | |
| Compound of | 4.79 ± 0.87 | −56.6 ± 7.9 | P <0.05 |
| Formula (Ia) | | | |
| (4 mg/kg, PO) | | | |
| Normal control, PO | 0.13 ± 0.13 | −98.9 ± 1.1 | P <0.05 |

Data represents Mean ± SEM

### 8 Appendix I

Individual Data for animal histological finding of cystic follicles after 4 weeks of Compound of Formula (Ia) treatment.

TABLE 8
Individual Data for Vehicle control group
Vehicle control-individual animal histopathological finding of cystic follicles

| Ovary Sections | Animal-1 | Animal-2 | Animal-3 | Animal-4 | Animal-5 | Animal-6 | Animal-7 | Animal-8 |
|---|---|---|---|---|---|---|---|---|
| 1 | 14 | 13 | 10 | 9 | 10 | 12 | 7 | 12 |
| 2 | 13 | 8 | 14 | 12 | 11 | 9 | 9 | 13 |
| 3 | 4 | 9 | 12 | 9 | 11 | 17 | 9 | 12 |
| 4 | 14 | 13 | 12 | 10 | 12 | 9 | 7 | 12 |
| 5 | 8 | 9 | 11 | 9 | 12 | 16 | 9 | 16 |
| 6 | 12 | 14 | 9 | 10 | 13 | 9 | 10 | 10 |
| Mean | 11.67 | 11.00 | 11.33 | 9.83 | 11.50 | 12.00 | 8.50 | 12.50 |

TABLE 9
Individual Data for Compound of Formula (Ia) (4 mg/kg) group
Compound of Formula I (a) (4 mg/kg)-Individual animal histopathological finding of cystic follicles

| Ovary Sections | Animal-9 | Animal-10 | Animal-11 | Animal-12 | Animal-13 | Animal-14 | Animal-15 | Animal-16 |
|---|---|---|---|---|---|---|---|---|
| 1 | 3 | 3 | 8 | 5 | 2 | 5 | 5 | 11 |
| 2 | 4 | 4 | 8 | 3 | 2 | 5 | 3 | 11 |
| 3 | 4 | 3 | 7 | 5 | 2 | 5 | 5 | 2 |
| 4 | 3 | 4 | 8 | 3 | 2 | 4 | 3 | 12 |
| 5 | 4 | 3 | 7 | 4 | 2 | 5 | 4 | 11 |
| 6 | 3 | 4 | 7 | 3 | 2 | 4 | 3 | 10 |
| Mean | 3.50 | 3.50 | 7.50 | 3.83 | 2.00 | 4.67 | 3.83 | 9.50 |

TABLE 10
Individual Data for Normal control group
Normal control-Individual animal histopathological finding of cystic follicles

| Ovary Sections | Animal-17 | Animal-18 | Anima1-19 | Animal-20 | Animal-21 | Animal-22 | Animal-23 | Animal-24 |
|---|---|---|---|---|---|---|---|---|
| 1 | 0 | 0 | 0 | 1 | 0 | 0 | 0 | 0 |
| 2 | 0 | 0 | 0 | 1 | 0 | 0 | 0 | 0 |
| 3 | 0 | 0 | 0 | 1 | 0 | 0 | 0 | 0 |
| 4 | 0 | 0 | 0 | 1 | 0 | 0 | 0 | 0 |
| 5 | 0 | 0 | 0 | 1 | 0 | 0 | 0 | 0 |
| 6 | 0 | 0 | 0 | 1 | 0 | 0 | 0 | 0 |
| Mean | 0.00 | 0.00 | 0.00 | 0.00 | 0.00 | 0.00 | 0.00 | 0.00 |

The efficacy of the compound of formula (Ia) in treating polycystic ovarian syndrome in subjects diagnosed with NAFLD is evaluated as follows:

### Example 4

### Objective:

The purpose of this study is to evaluate the efficacy and safety of Compound of Formula (Ia) 4 mg once daily in women with well characterized PCOS diagnosed with NAFLD.

### Primary Objective:

1. To evaluate the effect on hepatic fat content by proton-density fat-fraction (PDFF) by magnetic resonance imaging (MRI) of Compound of Formula (Ia) 4 mg once daily for 24 weeks vs placebo.

### Secondary Objectives:

To assess the effect of a 24-week treatment regimen of Compound of Formula (Ia) 4 mg on the following parameters in patients of PCOS with NAFLD:

1. Liver enzymes/LFTs.
2. Insulin resistance (IR) measured by Homeostasis Model Assessment (HOMA).
3. Liver stiffness measurement obtained via transient elastography/FibroScan®.
4. Controlled attenuation parameter obtained via transient elastography/FibroScan®.
5. Body weight, body mass index (BMI) and waist circumference.
6. MRI-derived total liver fat index and total liver volume.
7. Serum lipid profile and lipoproteins.
8. Sex hormone binding globulin.
9. Ovarian function.
10. Free androgen index.

### Criteria for Safety:

1. Vitals: blood pressure (BP) (sitting BP after 05 min rest; systolic and diastolic BP), pulse rate, oral temperature, respiratory rate (Visit 1 and Visit 3 to Visit 8).
2. Body mass index (BMI) at Screening Visit (Visit 1), at 12 weeks and end-of-treatment visit.
3. Waist measurements at Screening Visit (Visit 1), at 12 weeks and end-of-treatment visit.
4. The physical examination will consist of an evaluation of the head, neck, eyes, ears, nose, throat, chest, heart, lungs, abdomen, skin, extremities, neurological systems, musculoskeletal systems and weight measurement. Investigator should also evaluate the patients for hirsutism and virilizing signs (upper lip, chin, chest, upper abdomen, lower abdomen, thighs, back, arm and buttocks).

### 5. Laboratory Assessment

a. Hematology: Hematocrit, hemoglobin, mean corpuscular hemoglobin concentration (MCHC), mean corpuscular volume (MCV), platelet count, mean platelet volume, red blood cell (RBC) count, white blood cell (WBC) count, differential WBC count.
b. Liver enzymes/liver function tests (LFTs): aspartate aminotransferase (AST), alanine aminotransferase (ALT), alkaline phosphatase (ALP), total bilirubin (with conjugated bilirubin), gamma glutamyltransferase (GGT), serum protein and albumin.
c. Renal function tests (RFTs): blood urea nitrogen (BUN), creatinine, estimated glomerular filtration rate (eGFR) and uric acid. Estimated glomerular filtration rate will be calculated by using Chronic Kidney Disease Epidemiology Collaboration (CKD-EPI) equation.
d. Inflammatory marker: high sensitivity C-reactive protein (hs-CRP).
e. Lipid profile and lipoproteins: triglyceride (TG), total cholesterol (TC), high-density lipoprotein (HDL), low-density lipoprotein (LDL), small dense low-density lipoprotein (sdLDL), very low density lipoprotein (VLDL), free fatty acids, apolipoprotein A and apolipoprotein B.
f. Urine examination: physical examination (appearance, color, specific gravity and pH); microscopy (epithelial cells, red blood cells, pus cells, cast and crystals) and chemical examination (protein, glucose, bilirubin, urobilinogen, ketone bodies and nitrite).
g. T3, T4 and thyroid stimulating hormone (TSH)
h. Serum pregnancy test
i. Urine pregnancy
j. Serology: human immunodeficiency virus (HIV) type 1 and type 2, hepatitis A virus (HAV), anti-hepatitis B virus surface antigen (HBsAg) and hepatitis C virus (HCV) (at Visit 1).
k. International normalized ratio (INR) and prothrombin time (PT)
l. Cytokeratin 18
m. Free androgen index and Sex hormone binding globulin level
n. Total testosterone and free testosterone
o. Follicle stimulating hormone (FSH) and luteinizing hormone (LH) levels
p. 17-hydroxyprogesterone
q. Estradiol
r. Creatine Kinase (CK)
s. Cardiac function: 12-lead-electrocardiogram (ECG)
t. Glycemic control: Fasting plasma glucose (FPG), HbAlc and plasma insulin levels
6. Adverse event(s): Frequency and severity of adverse event (AE)/serious adverse events (SAEs), drop-outs due to AEs/SAEs for all subjects enrolled will be recorded. All AEs, will be assessed using Council for International Organizations of Medical Sciences (CIOMS) criteria using:
a. Causality
b. Severity
c. Seriousness
d. Expectedness

Note: Efficacy assessments and laboratory assessment (liver fat content by MRI-PDFF, controlled attenuation parameter, hormonal profile, fasting plasma glucose, lipid and lipoprotein profiles, MRI-derived total liver fat index and total liver volume, plasma insulin levels, homeostasis model assessment (insulin resistance [HOMA-IR]), liver stiffness by transient elastography/FibroScan®, ovarian function, liver enzymes/LFTs, hematology, coagulation test (PT/INR), renal function test, CK, hs-CRP, TNFα, uric acid, TSH, T3 and T4, serology, HIV type 1 and type 2, HAV, anti-HBsAg, HCV, serum pregnancy test, urine microscopy and urine chemistry) which will be done at Visit 1 will be considered for baseline visit.

### Criteria for Inclusion/Exclusion

### Inclusion Criteria:

1. Females, 18 to 45 years of age.
2. Previously confirmed diagnosis of PCOS by Rotterdam criteria (at least 2 of 3):
1) oligo-and/or anovulation;
2) hyperandrogenism (clinical and/or biochemical);
3) polycystic ovary morphology on ultrasonography (either 12 or more follicles measuring 2-9 mm in diameter or an increased ovarian volume>10 cm3).
3. Evidence of NAFLD within 6 months prior to the Screening Visit (Visit 1). The diagnosis of NAFLD is made according to the American Association for the Study of Liver Diseases (AASLD) criteria.
a) hepatic steatosis by imaging or histology,
b) no significant alcohol consumption,
c) no competing etiologies for hepatic steatosis, and
d) no co-existing causes for chronic liver disease.
4. Hepatic fat fraction≥10% by MRI-PDFF.
5. Willingness to participate in the study.
6. Ability to understand and give informed consent for participation.

### Exclusion Criteria:

1. Presence of other chronic liver diseases (hepatitis B or C, autoimmune hepatitis, cholestatic liver disease, Wilsons disease, hemochromatosis, etc.
2. Average alcohol consumption≥7 drinks per week for women in the 6 months prior to enrollment. Subjects will be required to have an “Alcohol Use Disorder Identification Test” (AUDIT) score of <8.
3. Clinical, imaging, or histological evidence of cirrhosis
4. Any of the following laboratory values:
a. Hemoglobin<9 g/dL
b. White blood cell count<2.5×103/μL
c. Neutrophil count<1.5×103/μL
d. Platelets<100×103/μL
e. Total Serum bilirubin>1.5 mg/dL (except in patient with known Gilbert Syndrome where TB up to 2.5 mg/dL is allowed)
f. Albumin<3.2 g/dL
g. Serum creatinine>1.5 mg/dL
h. Serum ALT or AST>250 IU/L
5. Patient with INR>1.3
6. Patients with chronic kidney disease and eGFR<40 ml/min
7. Ongoing use of medications known to cause hepatic steatosis (e.g., corticosteroids, amiodarone, methotrexate, tetracycline, tamoxifen, anabolic steroids, or valproic acid).
8. Prior bariatric surgery.
9. Weight loss of more than 5% in the 3 months preceding screening.
10. Severe co-morbidities (e.g., advanced cardiac, renal, pulmonary, or psychiatric illness).
11. Known allergy, sensitivity or intolerance to Compound of Formula (Ia), comparator or formulation ingredients (Saroglitazar Magnesium (Compound of formula I(a)) Micronized, Magnesium Oxide Light, Microcrystalline Cellulose, Anhydrous Lactose, Croscarmellose Sodium, Povidone k 30, Talc (luzena c), Colloidal Silicon Dioxide and Magnesium Stearate).
12. Use of antidiabetic and lipid lowering medications if the dose is not stable for at least the 3 months preceding screening.
13. Intake of Vitamin E (>100 IU/day) or multivitamins containing Vitamin E (>100 IU/day) 3 months before enrollment.
14. Use of drugs with potential effect on NAFLD/NASH such as S-adenosylmethionine (SAMe), Betaine, Pentoxifylline, Thiazolidinediones (Pioglitazone, Rosiglitazone), Obeticholic Acid or Milk Thistle in the 3 months prior to screening.
15. Changing doses of statins (Simvastatin, Pravastatin, Atorvastatin, Fluvastatin, Lovastatin, Rosuvastatin) or Fibrates (Clofibrate, Fenofibrate) in the 3 months prior to enrollment.
16. Illicit substance abuse within the past 12 months.
17. In women with child bearing potential: inability or unwillingness to practice contraception for the duration of the study.
18. Pregnant or breast feeding females.
19. Poorly controlled diabetes with HbA1c>8.5%.
20. Use of total parenteral nutrition in the 6 months preceding enrollment.
21. Women with known Cushing syndrome or hyperprolactinemia.
22. Late onset congenital adrenal hyperplasia, androgen-producing tumors.
23. Refusal or inability to comply with the requirements of the protocol, for any reason, including scheduled clinic visits and laboratory tests.

### Methodology:

This is a multicenter, phase 2A, randomized, double-blind, placebo-controlled study designed to evaluate the efficacy and safety of Compound of Formula (Ia) in women with well characterized PCOS, 18-45 years of age diagnosed with NAFLD according to the AASLD criteria within 6 months preceding the Screening Visit (Visit 1).
Eligible subjects will be screened within 4 weeks prior to randomization. During the Visit 1, subject will be seen by the investigator or designated study personnel and an AUDIT questionnaire will be administered. Inclusion and exclusion criteria will be verified during Visit 1 and Visit 3.
Eligible subjects will be enrolled into either of the two treatments arms: Compound of Formula (Ia) 4 mg tablets and matching placebo tablets in a 1:1 ratio.
The study will be conducted over a period of up to 34 weeks. Subjects will be evaluated at study sites for 8 scheduled visits: at screening visits (Visit 1: Day [−28], Visit 2: Day [−14 to −7]), randomization (Visit 3: Week 1, Day 1), Visit 4 (Week 2, Day 14), Visit 5 (Week 8, Day 56), Visit 6 (Week 12, Day 84), Visit 7 (Week 16, Day 112) Visit 8 (week 24, Day 168).

After completion of the study treatment period, the subjects will be followed for an additional period of 6 weeks without study medication until Visit 9 (week 30, Day 210).
Subjects will be monitored during the study and in every visit for development of any adverse events including drug induced liver injury. In addition to physical examination, laboratory data and ECGs done during the study period will be monitored to detect any treatment emergent adverse events.

### Screening Phase

Visit 1 (Day −28): Before each patient is admitted to the study, informed consent will be obtained from the patient (or her legally authorized representative) according to the regulatory and legal requirements of the participating country. Patient eligibility for participation in the study will be assessed. Current social, medical and ovarian dysfunction history will be obtained and physical examination, vital signs, ECGs and laboratory evaluations (including serology, hematology, lipid and lipoprotein profiles, liver enzymes/LFTs, hormonal profile and urinalysis) will be performed.
Patients will undergo a serum pregnancy test.
Visit 2 (Day −14 to −7): Liver function tests (AST, ALT, ALP, GGT, total protein, albumin, total bilirubin (with conjugated bilirubin) will be re-measured approximately 2 weeks from Day −28 to determine eligibility.

The variance in the levels of the repeat measures of serum ALT at Day −14 to −7 (Visit 2) must be <30%, compared to the Day −28 (Visit 1) levels to be eligible for study entry.

### Randomization and Treatment Phase

The randomization and treatment phase will include 6 outpatient visits (Visit 3 to Visit 8) over a period of 24 weeks including the randomization visit.
All the eligible subjects will be randomly assigned into either of the two treatments arms:
   Compound of Formula (Ia) 4 mg tablets and matching placebo tablets in a 1:1 ratio.
Efficacy assessments will be conducted at 12 and 24 weeks visits by measuring liver fat content by MRI-PDFF, hormonal profile, lipid and lipoprotein profiles, MRI-derived total liver fat index and total liver volume, homeostasis model assessment (insulin resistance [HOMA-IR]), liver stiffness by transient elastography/FibroScan®, controlled attenuation parameter, ovarian function and liver enzymes/LFTs.
In addition, subjects will be monitored during the study and at every visit for development of any adverse events including drug induced liver injury.
In addition to physical examination, laboratory data and ECGs done during the study period will be monitored to detect any treatment emergent adverse events.
   End-of-treatment Visit (Visit 8): An end-of-treatment visit will occur at 24 weeks for clinical assessment, reconciliation of study drug, and measurements of efficacy and safety endpoints.

Detailed efficacy and safety assessments are provided in Table 1 (Time and Events Schedule).

Safety follow-up visit (Visit 9): A final post-treatment visit will occur 6 weeks (±3 days) after the end-of-treatment visit for safety monitoring. A telephonic follow-up visit will be performed to assess any AE or SAE after the end-of-treatment visit for safety monitoring.

### Patient Withdrawal:

Patients may withdraw from the entire study at any time without penalty and for any reason without prejudice to her future medical care. A patient's withdrawal of consent and agreement to undergo a final examination will be documented on the electronic case report form. As far as possible, all assessments scheduled for end-of-treatment must be performed on all patients who receive the study drug but do not complete the study according to protocol.

Discontinuation of Patients from the Study or Study Drug:
A patient may be discontinued from the study for any of the following reasons:
Noncompliance to the protocol requirements.
Occurrence of a serious or intolerable AE.
The Sponsor or PI terminates the study.
Either the PI or the Sponsor decides that discontinuing the study or discontinuing the patient is in the patient's best interest.
For reasons related to safety.
The patient is lost to follow-up.
A patient may also be discontinued from study drug/study by the Sponsor, Regulatory Authorities or Institutional Review Board (IRB) or Independent Ethics Committee (IEC).
If the patient becomes pregnant.

### Concomitant Medications:

All the permitted and excluded concomitant medications will be recorded in this study.

### Total Enrollment:

Sixty patients are planned to be enrolled in this study. Patients will be randomly assigned to receive active drug (Compound of Formula (Ia) 4 mg) or placebo in a 1:1 ratio.

### Test Product:

### Dose:

### Mode of Administration:

Saroglitazar Magnesium (Compound of Formula (Ia)) tablet (Zydus) 4 mg Oral (once daily in the morning before breakfast)

### Reference/Placebo Therapy:

### Mode of Administration:

Placebo tablet
Oral (once daily in the morning before breakfast)
Duration of Treatment: 24 weeks

### Criteria for Efficacy

### Primary Efficacy Endpoint:

1. Change in hepatic fat content from baseline following 24 weeks of treatment as measured by MRI-PDFF.

### Secondary Efficacy Endpoints:

1. Changes from baseline to week 12 and week 24 in liver enzymes/LFTs: ALT, AST, ALP, GGT, serum protein, albumin and total bilirubin.
2. Changes from baseline to week 12 and week 24 in IR as measured by HOMA.
3. Changes from baseline to week 24 in markers of liver injury and fibrosis including CK-18, high sensitivity C-reactive protein (hs-CRP), tumor necrosis factor α (TNFα), and liver stiffness measured by transient elastography/FibroScan®.
4. Changes from baseline to week 24 in controlled attenuation parameter measured by transient elastography/FibroScan®.
5. Changes from baseline to week 12 and week 24 in body weight, BMI and waist circumference.
6. Changes from baseline to week 24 in MRI-derived measures of total liver fat index and total liver volume.
7. Changes from baseline to week 12 and week 24 in lipid and lipoprotein levels: TG, TC, HDL, LDL, sdLDL, VLDL, apolipoprotein A and apolipoprotein B.
8. Changes from baseline to week 12 and week 24 in sex hormone binding globulin (SHBG) level.
9. Changes from baseline to week 12 and week 24 in ovarian function (Total testosterone, 17-hydroxyprogesterone, free testosterone, luteinizing hormone, follicle-stimulating hormone, LH-to-FSH ratio and estradiol).
10. Changes from baseline to week 12 and week 24 in free androgen index.

### Criteria for Safety

1. Frequency and severity of AEs and serious AEs.
2. Clinical laboratory testing (hematology, clinical chemistry, hormonal profile and urinalysis).
3. Twelve-lead electrocardiogram.
4. Vital signs.
5. Physical examination.

### Statistical Methods:

General data analysis: Statistical Analysis Plan (SAP) will be prepared and finalized prior to database lock. The SAP will include detailed statistical aspects of the efficacy and safety analysis. Statistical analysis will be performed using SAS® software (version 9.4 or higher) (SAS Institute Inc., USA).

Demographic and baseline characteristics will be summarized by treatment and population set. Subject disposition and reason for withdrawal will be summarized and presented. Unless otherwise stated, all the continuous variables will be summarized using descriptive statistics such as n, mean, standard deviation, minimum, median and maximum. Categorical variables will be summarized using frequencies and percentages.

Efficacy analysis: All efficacy analysis will be based primarily on the Per Protocol (PP) population and analysis based on modified intent-to-treat (mITT) population will be considered as supporting analyses for this proof of concept study. The PP population will consist of all randomized patients completing the treatment phase and have not deviated from or violated the protocol in such a way that could affect efficacy outcome (i.e., had both baseline and end-of treatment MRI & has taken ≥80% of the drug). The mITT population will consist of all randomized patients who have taken at least one dose of the study treatment and have at least one post-baseline efficacy data. Last observation carried forward method will be used as an imputation method for post-baseline missing values for mITT analysis.
Primary efficacy analysis: The primary analysis for primary efficacy endpoint will be based on PP population. The primary efficacy endpoint in this study is the change in hepatic fat content from baseline following 24 weeks of treatment as measured by MRI-PDFF. The change from baseline of hepatic fat will be determined as (Week 24 value−Baseline value). The change from baseline at week 24 in hepatic fat content between treatments will be evaluated using analysis of covariance (ANCOVA) model with treatment as fixed effect and baseline value as covariate.

Treatment effects will be estimated using the least square means, standard error and 95% confidence interval (CI) from the ANCOVA model. The two treatment groups i.e., Compound of Formula (Ia) 4 mg versus Placebo, will be compared using difference in least-square means and pvalues from the ANCOVA model.

Secondary efficacy analysis: All secondary efficacy endpoints will be analyzed using the same statistical methods as primary efficacy endpoint.
Safety analysis: Safety analysis will be based on the safety population which will consist of all randomized patients who received at least 1 dose of study treatment with patients grouped according to the actual treatment received. All safety endpoints (AEs, clinical laboratory testing, ECGs, body weight, physical examination and vital signs) will be summarized by treatment group using the following descriptive statistics: N, mean, median, standard deviation, minimum and maximum for continuous variables and frequencies and percentages for categorical variables.

1. A pharmaceutical composition comprising a compound of formula (I) or a pharmaceutically acceptable salt thereof selected from Na⁺, K⁺, Ca⁺², and Mg⁺², for use in the treatment of polycystic ovarian syndrome (PCOS) in a patient in need of such treatment, wherein formula (I) is represented by

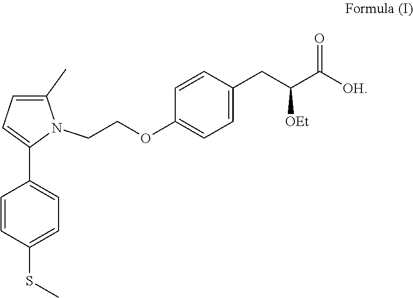 [SMILES: CCO[C@@H](Cc1ccc(OCCn2c(C)ccc2-c2ccc(SC)cc2)cc1)C(=O)O]

2. (canceled)

3. The pharmaceutical composition of claim 1, wherein the compound is Saroglitazar magnesium (formula (Ia));

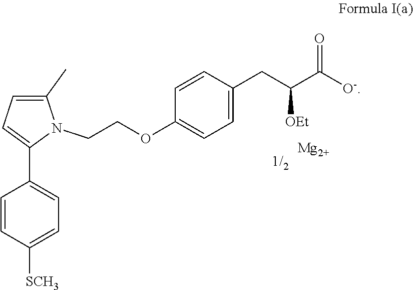

4. The pharmaceutical composition of claim 1, wherein the pharmaceutical composition further comprises one or more suitable pharmaceutically acceptable excipients selected from an additive, a stabilizer, a solubilizer, a diluent, a filler, a disintegrant, a binder, a lubricant, a glidant, a wetting agent, and a solvent.

5. (canceled)

6. The pharmaceutical composition of claim 1, wherein the pharmaceutical composition is administered orally to the patient in the form of a tablet or a capsule.

7. (canceled)

8. The pharmaceutical composition of claim 1, wherein the pharmaceutical composition provides the compound of formula (I) or the pharmaceutically acceptable salt thereof at a dose in the range of 0.5 mg to 10 mg with respect to the weight of the compound of formula (I).

9. The pharmaceutical composition of claim 3, wherein the pharmaceutical composition is for administration to a human subject to provide Saroglitazar magnesium at a dose of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg or 10 mg.

10. (canceled)

11. (canceled)

12. The pharmaceutical composition of claim 1, wherein the use is considered in terms of the treatment or amelioration of polycystic ovarian syndrome in a human subject.

13. The pharmaceutical composition of claim 1, wherein the use is considered in terms of the treatment or amelioration of polycystic ovarian syndrome in a human subject diagnosed with non-alcoholic fatty liver disease (NAFLD).

14. The pharmaceutical composition of claim 1, wherein the pharmaceutical composition is co-administered with an additional therapeutic agent selected from metformin, an SGLT2 inhibitor, and a DPP-IV inhibitor.

15. (canceled)

16. The pharmaceutical composition of claim 14, wherein the DPP-IV inhibitor is selected from vildagliptin, sitagliptin, teneligliptin, saxagliptin, and linagliptin, and the SGLT2 inhibitor is selected from canagliflozin, dapagliflozin, empagliflozin, luseogliflozin, and ipragliflozin.

17. A method of treating polycystic ovarian syndrome (PCOS) in a subject in need thereof, the method comprising administering to the subject an effective amount of a compound of formula (I) or a pharmaceutically acceptable salt thereof selected from Na⁺, K⁺, Ca⁺², and Mg⁺², or a pharmaceutical formulation thereof, wherein formula (I) is represented by

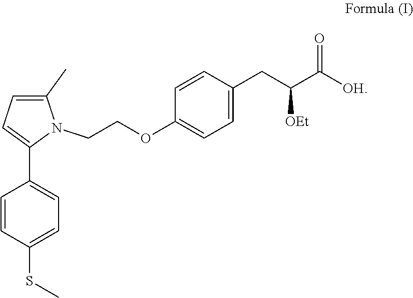 [SMILES: CCO[C@@H](Cc1ccc(OCCn2c(C)ccc2-c2ccc(SC)cc2)cc1)C(=O)O]

18. (canceled)

19. The method of claim 17, wherein the compound is Saroglitazar magnesium (formula (Ia): 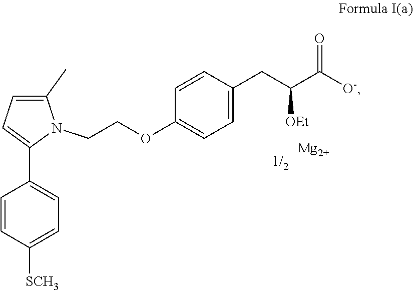
or a pharmaceutical formulation thereof.

20. (canceled)

21. The method of claim 17, wherein the pharmaceutical formulation comprises one or more suitable pharmaceutical excipients selected from an additive, a stabilizer, a solubilizer, a diluent, a filler, a disintegrant, a binder, a lubricant, a glidant, a wetting agent, and a solvent.

22. (canceled)

23. The method of claim 17, wherein the compound of formula (I) or a pharmaceutical formulation thereof, is administered orally to the subject in the form of a tablet or a capsule.

24. (canceled)

25. The method of claim 17, wherein the compound of formula (I) or a pharmaceutically acceptable salt thereof, is administered to the subject at a dose in the range of 0.5 mg to 10 mg with respect to the weight of the compound of formula (I).

26. The method of claim 19, wherein the Saroglitazar magnesium or a pharmaceutical formulation thereof is administered to a human subject to provide Saroglitazar magnesium at a dose of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg or 10 mg.

27. (canceled)

28. (canceled)

29. The method of claim 17, wherein treatment is carried out for at least 1 week to 24 weeks.

30. The method of claim 17, wherein the compound of formula (I) or pharmaceutically acceptable salt thereof, or pharmaceutical formulation thereof is co-administered with an additional therapeutic agent selected from metformin, an SGLT2 inhibitor, and a DPP-IV inhibitor.

31. (canceled)

32. The method of claim 30, wherein the DPP-IV inhibitor is selected from vildagliptin, sitagliptin, teneligliptin, saxagliptin, and linagliptin, and the SGLT2 inhibitor is selected from canagliflozin, dapagliflozin, empagliflozin, luseogliflozin, and ipragliflozin.

33. The method of claim 17, wherein treatment results in an increase in the level of serum FSH, and a decrease in the level of serum testosterone and LH/FSH ratio.

34. A method of treating polycystic ovarian syndrome (PCOS) in a subject diagnosed with NAFLD, the method comprising administering to the subject an effective amount of a compound of formula (I) or a pharmaceutically acceptable salt thereof selected from Na⁺, K⁺, Ca⁺², and Mg⁺², or a pharmaceutical formulation thereof, wherein formula (I) is represented by

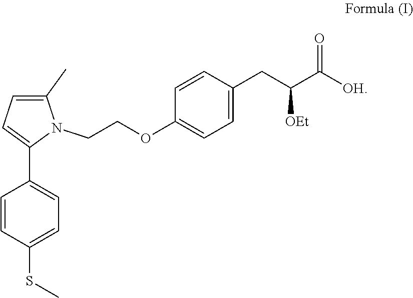 [SMILES: CCO[C@@H](Cc1ccc(OCCn2c(C)ccc2-c2ccc(SC)cc2)cc1)C(=O)O]

35. (canceled)

36. The method of claim 34, wherein the compound is Saroglitazar magnesium (formula (Ia)): 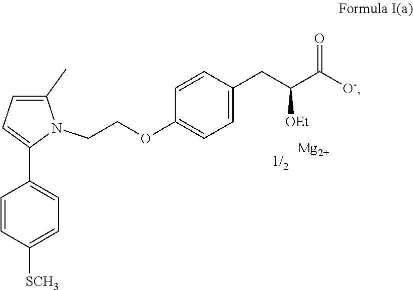
or a pharmaceutical formulation thereof.

37. (canceled)

38. The method of claim 34, wherein the pharmaceutical formulation comprises one or more pharmaceutical excipients selected from an additive, a stabilizer, a solubilizer, a diluent, a filler, a disintegrant, a binder, a lubricant, a glidant, a wetting agent, and a solvent.

39. (canceled)

40. The method of claim 34, wherein the compound of formula (I), or the pharmaceutically acceptable salt thereof, or the pharmaceutical formulation thereof is administered to the subject orally in the form of a tablet or a capsule.

41. (canceled)

42. The method of claim 34, wherein the compound of formula (I) or the pharmaceutically acceptable salt thereof is administered to the subject at a dose in the range of 0.5 mg to 10 mg.

43. The method of claim 36, wherein the Saroglitazar magnesium or a pharmaceutical formulation thereof is administered to a human subject to provide Saroglitazar magnesium at a dose of 0.5 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg or 10 mg.

44. (canceled)

45. (canceled)

46. The method of claim 34, wherein treatment is carried out for at least 1 week to 24 weeks.

47. The method of claim 34, wherein the compound of formula (I), pharmaceutically acceptable salt thereof, or pharmaceutical formulation thereof, is co-administered with additional therapeutic agent selected from metformin, an SGLT2 inhibitor and a DPP-IV inhibitor.

48. (canceled)

49. The method of claim 47, wherein the DPP-IV inhibitor is selected from vildagliptin, sitagliptin, teneligliptin, saxagliptin, and linagliptin, and the SGLT2 inhibitor is selected from canagliflozin, dapagliflozin, empagliflozin, luseogliflozin, and ipragliflozin.

50. The method of claim 34, wherein treatment results in an increase in the level of serum FSH, and a decrease in the level of serum testosterone and LH/FSH ratio.
